(19) United States
(12) Patent Application Publication
(10) Pub. No.: US 2011/0282185 A1
(43) Pub. Date: Nov. 17, 2011

(54) Gasket for Pre-Filled Syringe and Pre-Filled Syringe

(75) Inventors: Kazuyuki YANASE, Saitama (JP); Keizou NAKAMOTO, Saitama (JP); Kazumi IIJIMA, Gumma (JP); Fumiko IIJIMA, Gumma (JP); Hiromi Suzuki, Chiba (JP)

(73) Assignee: BRACCO SUISSE S.A., Manno (CH)

(21) Appl. No.: 13/111,597

(22) Filed: May 19, 2011

Related U.S. Application Data
(63) Continuation-in-part of application No. 09/720,762, filed on Dec. 28, 2000, now abandoned.

(30) Foreign Application Priority Data
Jun. 29, 1998 (JP) 1998000183005

(51) Int. Cl. A61B 5/055; A61B 6/00

(52) U.S. Cl. 600/420; 600/432

(57) ABSTRACT

The present invention relates to a gasket (7) for a pre-filled syringe 1 into which liquid (3) is charged, wherein a peripheral side surface of the gasket (7 a) that is in contact with an inner surface of the syringe barrel (2) is provided with a restriction (8), the bottom surface (7c) of the gasket (7) that is not in contact with the liquid (3) is formed into a tapered shape (10) and the gasket (7) has a hardness comprised from 57 to 60 when measured by a JIS hardness meter.

### CROSS-REFERENCE TO RELATED APPLICATIONS

This application is a continuation-in-part (CIP) of co-pending U.S. application, U.S. Ser. No. 09/720,762, filed Dec. 28, 2000, which is a U.S. national phase application of corresponding international application number PCT/JP1999/03459 filed Jun. 29, 1999, which in turn claims priority to and the benefit of the Japanese application number 1998000183005, filed Jun. 29, 1998, the contents of each of which all are incorporated herein by reference in their entireties.

### FIELD OF THE INVENTION

The present invention relates to a gasket for a pre-filled syringe and the pre-filled syringe.

### BACKGROUND ART

In general, liquid is charged into a syringe barrel when it is used. In recent years, a pre-filled syringe in which liquid (such as, for example a chemical or pharmaceutical) is previously charged into the syringe barrel has been developed, and reduced the work load in medical sites. Recently, a pre-filled syringe in which contrast medium is charged into the syringe is also used. Since the contrast medium can have a relatively high viscosity, it is important to increase the sliding ability of the gasket in such pre-filled syringes so as to reduce the charging pressure. Especially when a syringe barrel is made of polymeric resin, since the sliding ability of the gasket is inferior, silicon oil is applied onto a peripheral side surface of a gasket that is in contact with an inner surface of the syringe barrel to enhance the sliding ability, or polyethylene fluoride resin such as Teflon (registered trade name) is laminated. However, if silicon applied to a gasket enters the liquid contents of the syringe, it becomes foreign matter which may cause product defect, and there is apprehension that such foreign matter may have toxicity to the human body.

It is normal that the pre-filled syringe is sterilized after the liquid is charged or if liquid is charged in axenic conditions. Sterilization after charging is carried out, for example, by heating the pre-filled syringe, but it is important to form the gasket into such a shape that high pressure liquid caused by heat at the time of sterilization is not leaked. When polyethylene fluoride resin or the like is laminated on a gasket, since the polyethylene fluoride resin is hard, if the gasket is inserted into the syringe barrel, fine wrinkles may be generated on a peripheral side surface of the gasket, and liquid may be leaked through the wrinkles at the time of sterilization.

The present invention provides a solution to these problems.

### SUMMARY OF THE INVENTION

In one embodiment, there is provided a gasket used for a pre-filled syringe into which liquid is charged, wherein a peripheral side surface of the gasket that is in contact with an inner surface of the syringe barrel is provided with a restriction, and a periphery of a bottom surface of the gasket that is not in contact with the liquid is formed into a tapered shape.

In this gasket, it is preferable that hardness of the gasket is 55 to 60 when the hardness is measured by a JIS hardness meter.

Further, it is preferable that the peripheral side surface that is in contact with the inner surface of the syringe barrel and/or a bottom surface that is in contact with liquid is laminated with polyethylene fluoride resin.

In another embodiment there is provided a pre-filled syringe into which liquid is charged and tightly closed with the gasket described above. In this pre-filled syringe, the liquid is, preferably a contrast medium, preferably selected from an iodinated X-ray contrast medium and a paramagnetic NMR contrast agent. Iodinated contrast agents are preferably selected from the group consisting of: Iomeron®, Omnipaque®, and Ultravist®. Paramagnetic contrast agents are preferably selected from the group consisting of: ProHance® MultiHance®, Dotarem® and Magnevist®.

In a preferred embodiment, the gasket plays a role as a lid for tightly closing the liquid charged into the syringe barrel, and also plays a role as a piston when the pre-filled syringe is used. As the JIS hardness meter for measuring the hardness of the gasket, “Durometer” produced by Shimazu Seisakusho can be used for example. A preferable range of the hardness of the gasket measured by the JIS hardness meter is 57-60 more preferably 57 to 59. Even more preferred is a JIS hardness of 57 measured by a JIS hardness meter.

The material of the gasket is limited only to an appropriate hardness, but preferred materials include normal butyl rubber, silicon rubber or polymeric resin, and more preferably, chlorinated butyl rubber or chlorinated butyl rubber based material.

The peripheral side surface of the gasket that is in contact with the syringe barrel or the bottom surface that is in contact with liquid can be laminated with polyethylene fluoride resin using a conventionally known laminating method. Silicon may be applied to the peripheral side surface of the gasket that is in contact with the inner surface of the syringe barrel, but it is preferable that silicon is not applied to the bottom of the gasket that is in contact with the liquid charged into the syringe barrel. The silicon can also be applied by a conventionally known application method.

The material of the syringe barrel is not limited, and any glass or resin can be used. Resin is especially preferable, such as, for example, cyclo-polyolefin fiber.

The liquid to be charged into the syringe barrel is not limited to contrast medium, and other liquids including pharmaceuticals may be used. An example of a contrast medium is iomeprole. The amount of liquid to be charged into the syringe barrel can vary, but is usually about 10 to 200 ml, and more preferably 50 to 100 ml.

### BRIEF DESCRIPTION OF THE DRAWINGS

FIG. 1 is a sectional view of a pre-filled syringe according to an embodiment of the present invention; and

FIG. 2 is a side view of a gasket according to the embodiment of the invention.

### DETAILED DESCRIPTION

A preferred embodiment of the present invention will be explained with reference to the accompanying drawings below.

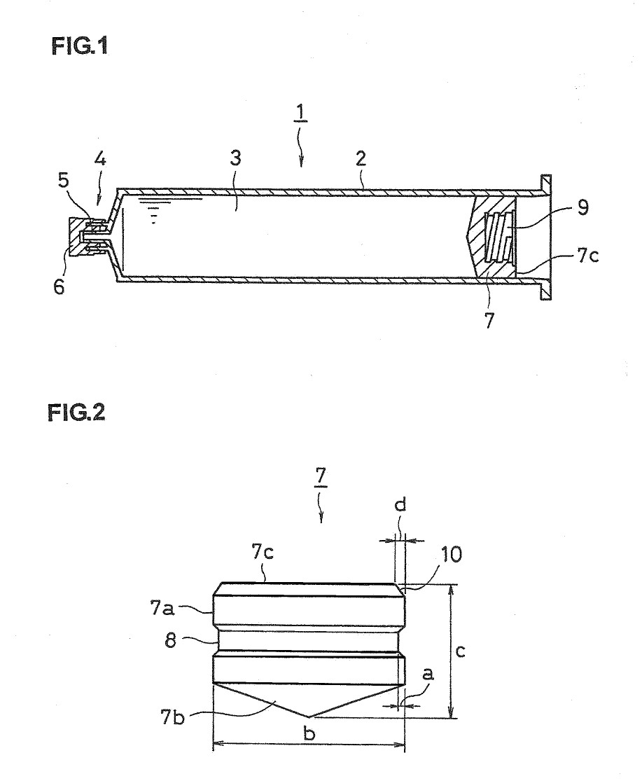

As shown in FIG. 1, in a pre-filled syringe 1 of this embodiment, a contrast medium 3 as one example of a liquid is charged in a cylindrical syringe barrel 2. The syringe barrel 2 is made of a suitable material such as annular polyolefin fiber for example. The syringe barrel 2 is provided at its tip end (left end of the syringe barrel 2 in FIG. 1) with a nozzle 4. The nozzle 4 is formed with a luer lock 5, and by mounting a cap 6 on the luer lock 5, the nozzle 4 is tightly closed. A cylindrical gasket 7 is inserted into the syringe barrel 2 from an opened rear end (right end of the syringe barrel 2 in FIG. 1) of the syringe barrel 2, and the liquid (contrast medium) 3 in the syringe barrel 2 is tightly closed by the gasket 7.

The gasket 7 is made of normal butyl rubber, silicon rubber or polymeric resin, more preferably, chlorinated butyl rubber or chlorinated butyl rubber based material. The gasket 7 preferably has a hardness of 55 to 60 degrees, preferably 57 to 60 even more preferably 57 to 59 degrees when the hardness is measured using JIS hardness meter. Even more preferred is a JIS hardness of the gasket of about 57 measured by a JIS hardness meter.

As shown in FIG. 2, a peripheral side surface 7a of the gasket 7 that is in contact with an inner surface of the syringe barrel 2 is formed with an annular restriction 8. The dimensions of the gasket and the annular restriction may vary and will be apparent to the skilled artisan from the size of the syringe, etc. However, a depth a of the restriction 8 may be about 0.05 to 1.0 mm, and more preferably, 0.1 to 0.5 mm if the gasket has a diameter b of 30 to 35 mm and a height c of 15 to 18 mm. Thus according to a preferred embodiment, the ratio between the inner and the outer diameter of the restriction (8 in FIG. 2) is comprised from 0.9 to 1 even more preferably comprised from 0.93 to 0.98.

A bottom surface 7b (lower surface of the gasket 7 in FIG. 2) that is in contact with the liquid (contrast medium) 3 charged into the syringe barrel 2 is formed into a conical surface. The bottom surface 7b and the peripheral side surface 7a are preferably laminated with a synthetic resin, preferably a polyethylene fluoride resin. A central portion of a bottom surface 7c (upper surface of the gasket 7 in FIG. 2) that is not in contact with the liquid (contrast medium) 3 charged into the syringe barrel 2 is formed, as shown in FIG. 1, with a threaded hole 9 into which a rod is fitted. As shown in FIG. 2, a periphery of the bottom surface 7c is formed into a tapered slant 10. A range (range of the gasket 7 from an outer diameter in its diametrical direction) d) where the slant 10 is formed is about 0.5 to 5 mm, and more preferably about 1 to 3 mm if the gasket has a diameter b) of 30 to 35 mm and a height c) of 15 to 18 mm.

According to this embodiment pre-filled syringe 1 having the above-described structure, the cap 6 is removed from the nozzle 4, and a means of administration, such as a needle or tip end of an extension or patient administration tube (not shown), for example, is threadedly fitted to the luer lock 5. A rod or other piston (not shown) is fitted to the bottom surface 7c of the gasket 7. Then, the rod is pushed to push out the liquid (contrast medium) 3 in the syringe barrel 2 through the administration means (e.g. extension tube), thereby charging the contrast medium 3 into a target position. The syringe is preferably filled with a contrast medium wherein said contrast medium is an iodinated X-ray contrast medium or a paramagnetic NMR contrast agent. Iodinated contrast agents are preferably selected from the group consisting of: Iomeron®, Omnipaque®, and Ultravist®. Paramagnetic contrast agents are preferably selected from the group consisting of: ProHance® MultiHance®, Dotarem® and Magnevist®.

According to this embodiment the syringe is pre-filled with the liquid (contrast media) and is then sterilized. Then, the rod is pushed to push out the liquid 3 in the syringe barrel 2 through the administration means (e.g. extension tube), thereby charging the liquid (contrast medium) 3 into a target position.

### INDUSTRIAL APPLICABILITY

According to the invention, the sliding ability, especially when polyolefin resin is used as material of a syringe barrel is used is remarkably enhanced, and liquid does not leak almost at all when the pre-filled syringe is sterilized. The gasket can smoothly move at the time of sterilization even when the gasket is pushed out, and there is no adverse possibility that the gasket is diagonally inclined with respect to a center axis of the syringe barrel.

### DESCRIPTION OF CHARACTERS IN FIGS. 1 AND 2

1 pre-filled syringe
2 syringe barrel
3 liquid, such as contrast medium
4 nozzle
5 luer lock
6 cap
7 gasket
7a peripheral side surface
7b, 7c bottom surface
8 annular restriction
9 threaded hole
10 tapered slant

### EXPERIMENTAL SECTION

In order to identify the optimal hardness of the gasket according to the invention, gaskets of two different hardnesses were developed and tested when Teflon-laminated. Each gasket was tested in a syringe barrel filled with either a contrast medium (IOM 300 mg) or distilled water (WFI: water for injection) and sterilized at 115° C. for 60 minutes. After drying, multiple tests were performed on each gasket and syringe, including a set of three tests that checked:

(1) gasket position;
(2) the relative alignment of the gasket within the syringe (“crook”); and
(3) the presence or absence of liquid leakage.

The gasket position was determined by measuring the distance from a collar surface of the syringe barrel to a gasket screw-side bottom surface using a gasket position inspector, a table-type gasket inspector, or a digital caliper. For 100 mg syringes, gaskets having gasket positions between 7.3 and 11.5 mm were considered acceptable, but gaskets positions measured at less than 7.3 mm were considered to have failed the test. For 50 mg syringes, gasket positions between 69.0 and 74.0 mm were considered acceptable, but positions measured at less than 69.0 mm were considered to have failed the test.

The crook of the gasket was determined by measuring the maximum value and the minimum value of the measurement result of the gasket position at each position of the bottom surface, the difference between these values being defined as the value of the crook. Gaskets having crook values of 2 mm or less were considered acceptable and those with a value greater than 2 mm were considered to have failed the test.

With respect to testing the presence or absence of leakage, it was visually observed whether or not the contrast medium was leaking in a droplet behind a contact part between the gasket and the syringe barrel. Those gaskets with no observed leakage were considered acceptable and those with observable liquid leakage were considered to have failed the test.

Table 1 shows the results of the tests using gaskets with two different JIS hardness: a hardness of JIS 48-51 and a hardness of JIS 57-60.

Table 2 shows the results of gaskets with a JIS hardness of 57-59, but varying whether the liquid contact portion of the gasket was treated with silicone (“B2-41”) or not (“B2-01”) and the liquid used for filling the syringe.

The gasket having a JIS hardness of 57-60 showed better results when no silicon treatment was carried out.

In summary, Table 1 shows the testing results of the selected gasket shape made of rubber, with one set of gaskets having a JIS hardness of 48-51 and the second set having a JIS hardness of 57-60. The same results were confirmed with both 100 mg and 50 mg syringes.

TABLE 1
Comparison of Test Results
Gasket Shape according to the present invention and
Rubber JIS hardness: 57-60 or 48-51

| | | | Gasket position | | Crook | | Leakage | |
|---|---|---|---|---|---|---|---|---|
| | | No. | Test | | Test | | Test | |
| Test | JIS | Gaskets | Number | | Number | | Number | |
| No. | Hardness | Tested | Rejected | % | Rejected | % | Rejected | % |
| 1 | 48-51 | 198 | 98 | 49.5 | 2 | 1.0 | 100 | 25.8 |
| 5 | 57-60 | 190 | 0 | 0 | 0 | 0 | 0 | 0 |

As shown above, almost half (49.5%) of the gaskets with a JIS hardness of 48-51 were rejected for failing the position test, 1% for failing the crook test, and 25.8% for failing the leakage test. It was observed that many of the gaskets failed due to leakage or the changing of the gasket position within the syringe (leading to failure of the gasket position and/or the crook test) that occurred during the sterilization process.

In contrast, none of the gaskets with a JIS hardness of 57-60 were rejected for failing the gasket position, crook or leakage tests. Such a dramatic improvement represents an unexpected result.

These results (i.e. no gasket rejected under the: 1) gasket position, 2) Crook Test or 3) Leakage Test) were confirmed for rubber gaskets of the invention having a JIS hardness of 57-59 and no silicon treatment, as shown in Table 2. When the gaskets of the invention were silicon treated, leakage was observed in just 1% of the cases. Similar results were obtained with syringes loaded with liquids having different densities such as Iomeron (IOM) and water (WFI).

TABLE 2

| | | | Gasket Material; Hardness; 57-59 | | | | | Filling fluid: WFI or IOM300 mg | | | | |
|---|---|---|---|---|---|---|---|---|---|---|---|---|
| | | | Reason of | | | Gasket | | Volume; 102 ml | | | | |
| | Type of | Number of | Rejection | | | Position | Crook [mm] | | | | Droplet | |
| liquid | gasket | Sample | 1) 2) | Number | % | MIN | MIN | MAX | Average | Stdev | Number | % |
| WFI | EP-15-E | 40 | Abnomal position | 0 | 0.0 | 8.01 | 0.33 | 1.03 | 0.61 | 0.16 | 0 | 0.0 |
| | B2-41⁽³⁾ | | Crooked gasket | 0 | 0.0 | | | | | | | |
| | | | Total | 0 | 0.0 | | | | | | | |
| IOM | EP-15-E | 99 | Abnomal position | 0 | 0.0 | 7.79 | 0.04 | 0.82 | 0.38 | 0.16 | 0 | 0.0 |
| | B2-01 | | Crooked gasket | 0 | 0.0 | | | | | | | |
| | | | Total | 0 | 0.0 | | | | | | | |
| IOM | EP-15-E | 99 | Abnomal position | 0 | 0.0 | 8.26 | 0.10 | 1.14 | 0.41 | 0.22 | 1 | 1.0 |
| | B2-41 | | Crooked gasket | 0 | 0.0 | | | | | | | |
| | | | Total | 0 | 0.0 | | | | | | | |

1) Abnormal position: less than 7.3 mm of total length between tale of barrel position and gasket's bottom position
2) Crook: ≦2 mm OK; NG > 2 mm
⁽³⁾Liquid contact portion of the gasket treated with silicone (“B2-41”) or not (“B2-01”).

We claim:

1. A gasket for a pre-filled syringe into which liquid is charged, wherein said gasket comprises:
a peripheral side surface of the gasket that is in contact with an inner surface of the syringe barrel, provided with at least one restriction, wherein said restriction has an inner diameter with a ratio comprised from 0.93 to 0.98 of the gasket's diameter; and
a periphery of a bottom surface of the gasket that is not in contact with the liquid formed into a tapered shape;
wherein the hardness of the gasket is comprised from 55 to 60, when the hardness is measured by a JIS hardness meter.

2. The gasket according to claim 1 wherein the peripheral side surface that is in contact with an inner surface of the syringe barrel is laminated with a synthetic resin.

3. The gasket according to claim 2 wherein a bottom surface of the gasket that is in contact with liquid is laminated with a synthetic resin.

4. The gasket according to any one of claim 2 or 3 wherein said synthetic resin is polyethylene fluoride.

5. The gasket according to claim 1 wherein the hardness is comprised from 57 to 60.

6. A pre-filled syringe into which liquid is charged, comprising a gasket according to any one of claims 1-3.

7. The pre-filled syringe according to claim 6, wherein said liquid is a contrast medium.

8. The pre-filled syringe according to claim 7 wherein said contrast medium is selected from the group consisting of iodinated or paramagnetic contrast agents.

9. The pre-filled syringe according to claim 8, wherein the contrast medium is selected from the group consisting of Iomeron®, ProHance® and MultiHance®.
